(19) United States
(12) Patent Application Publication
(10) Pub. No.: US 2008/0213325 A1
(43) Pub. Date: Sep. 4, 2008

(54) POWDER MIXTURE CONSISTING OF TITANIUM DIOXIDE, ZINC OXIDE AND ZINC/TITANIUM MIXED OXIDE

(75) Inventors: Kai Schumacher, Hofheim (DE); Steffen Hasenzahl, Maintal (DE); Martin Moerters, Rheinfelden (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Degussa AG, Duesseldorf (DE)

(21) Appl. No.: 12/100,746

(22) Filed: Apr. 10, 2008

Related U.S. Application Data
(62) Division of application No. 10/530,708, filed on Apr. 8, 2005, now abandoned.

(30) Foreign Application Priority Data
Dec. 20, 2002 (DE) 102 59 860.6

(51) Int. Cl. A61K 8/02; A61Q 19/00

(52) U.S. Cl. 424/401

(57) ABSTRACT

A powder mixture consisting of zinc/titanium mixed oxide particles, titanium dioxide particles and zinc oxide particles, wherein the zinc/titanium mixed oxide particles have a composition according to the formula (ZnO)₁₋ₓ(TiO₂)ₓ, where 0.01<x<0.99, and are obtained from a thermal process and the powder mixture exhibits remission which, in the UV range from 320 to 400 nm, is lower than that of titanium dioxide and, in the UV range below 320 nm, is lower than that of zinc oxide.

The powder mixture may be produced by introducing into a flame, as is used for the production of pyrogenic metal oxides by means of flame hydrolysis, an aerosol of a zinc compound, homogeneously mixing said aerosol with the flame hydrolysis gas mixture, allowing the aerosol/gas mixture to react in a flame, separating the solid reaction products from the stream of gas and then performing thermal treatment.

The powder mixture may also be produced by dispersing titanium dioxide powder in the presence of a solution of a zinc compound, wherein the ratio of titanium dioxide and zinc salt corresponds to the subsequently desired ratio of titanium dioxide and zinc oxide in the final product, then removing the solvent by evaporation and thermally treating the residue.

The powder mixture may be used in sunscreen preparations.

### CROSS REFERENCE TO RELATED APPLICATIONS

This application is a division of U.S. patent application Ser. No. 10/530,708, which is the U.S. national stage of International Application No. PCT/EP03/13923, filed Dec. 9, 2003, the disclosures of which are incorporated herein by reference in their entireties. This application claims priority to German Patent Application No. 102 59 860.6, filed Dec. 20, 2002, the disclosure of which is incorporated herein by reference in its entirety.

### BACKGROUND

Cosmetic preparations, such as creams or lotions, containing UV filters, which are largely transparent on the skin and are pleasant to use, are used to protect the skin from excessively high intensity UV radiation.

The UV filters they contain comprise one or more organic compounds which absorb within the wavelength range between 290 and 400 nm: UVB radiation (290 to 320 nm), UVA radiation (320 to 400 nm).

The higher-energy UVB radiation causes the symptoms typical of sunburn and is also responsible for suppressing the immune defences, while UVA radiation, which penetrates into the deeper skin layers, causes premature skin ageing. Since the interaction of the two types of radiation can apparently promote the occurrence of dermatological conditions caused by light, such as skin cancer, efforts have long been made to achieve further significant improvements in existing levels of UV protection.

It is known that microfine (ultrafine) pigments based on metal oxides can scatter, reflect and absorb UV radiation. They are accordingly an effective complement to the organic UV filters in sunscreen preparations.

Microfine titanium dioxide is widely used in cosmetic formulations because it is chemically inert and toxicologically safe and causes neither skin irritation nor sensitisation. In addition to titanium dioxide, microfine zinc oxide is also used.

Zinc oxide has long been widely used in pharmaceutically active skin preparations such as powders, ointments, creams and lotions. In cosmetic products, zinc oxide, like titanium dioxide, is used in decorative preparations due to its opacifying and brightening properties. It is for the same reasons that pigment grade zinc oxide has not hitherto become as well established in use in sunscreens as have titanium dioxide pigments. Conventional zinc oxide pigment provides opaque white coverage on surfaces. Zinc oxide has a relatively high refractive index of approx. 2.0. If transparent forms are to be obtained, micronised zinc oxide particles must be used, as is the case with titanium dioxide. Microfine zinc oxide generally has a particle size of 10 to 100 nm and a specific surface area of approx. 10 bis 70 m²/g. Its action extends over the entire UV range, i.e. from UVA radiation via UVB radiation up to UVC. Zinc oxide with a relatively sharp UVA absorption edge at 370 nm absorbs better than titanium dioxide in the UVA range.

Particular problems arise if it is intended to use zinc oxide and titanium dioxide simultaneously in a sunscreen preparation. Such a combination is entirely sensible as zinc oxide absorbs more strongly in the UVA range and titanium dioxide more strongly in the UVB range, which would mean that broadband absorption could be achieved over the entire UV range. However, the two substances have different isoelectric points: TiO₂ approx. 5 to 6 and ZnO approx. 9.5. At a pH value of between 5 and 7, which is typical for cosmetic products, oppositely charged particles may be present, which attract each other and may result in agglomeration or flocculation. This risk primarily occurs when both metal oxides are present in the aqueous phase.

### SUMMARY

The object of the present invention was accordingly to overcome the existing disadvantages of combined use of titanium dioxide and zinc oxide and to provide a powder which combines the advantages of zinc oxide and titanium dioxide.

The invention provides a powder mixture consisting of zinc/titanium mixed oxide particles, titanium dioxide particles and zinc oxide particles, wherein the zinc/titanium mixed oxide particles have a composition according to the formula (ZnO)₁₋ₓ(TiO₂)ₓ, where 0.01<x<0.99, and are obtained from a thermal process and wherein the powder mixture exhibits remission which, in the UV range from 320 to 400 nm, is lower than that of titanium dioxide and, in the UV range below 320 nm, is lower than that of zinc oxide.

There are no further restrictions as to the origin of the titanium dioxide and zinc oxide particles. They may originate from thermal or pyrogenic processes, sol/gel processes or precipitation processes.

It is only the zinc/titanium mixed oxide particles for the purposes of the invention which originate from a thermal process. A thermal process may, on the one hand, be taken to involve the conversion of zinc and titanium starting compounds at elevated temperatures. According to the invention, thermal processes may, on the other hand, be taken to comprise pyrogenic processes with subsequent heat treatment of the reaction mixture. A pyrogenic process may be taken to comprise flame hydrolysis or flame oxidation of metal or metalloid compounds in the gas phase in a flame, produced by the reaction of a fuel gas, preferably hydrogen, and oxygen. In such a process, highly disperse primary particles are initially formed which, as the reaction proceeds, may combine to form aggregates and the latter may in turn further congregate to form agglomerates. The BET surface area of these primary particles generally has a value between 5 and 600 m²/g.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing, wherein:

FIG. 1 shows the remission (in %) of a powder from Example 1 (denoted I) and Example 2C (II) in comparison with a pyrogenically produced titanium dioxide (P25, Degussa, III) and a zinc oxide (Nanox 100, Elementis, IV) as a function of wavelength.

### DETAILED DESCRIPTION OF EXEMPLARY EMBODIMENTS

It is known to produce zinc/titanium mixed oxide by a pyrogenic process as described in EP-A-1138632. It has, however, been found that, at the desired high zinc oxide contents (>20 wt. %), a non-homogeneous product mixture is obtained which is unsuitable for cosmetic purposes. The product produced according to EP-A-1138632 with a zinc oxide content of approx. 20 wt. % may thus be used for the purposes of the invention.

It is an essential feature of the invention that, in order to achieve remission which, in the UV range from 320 to 400 nm, is lower than that of titanium dioxide and, in the UV range below 320 nm, is lower than that of zinc oxide, the zinc/titanium mixed oxide particles originate from a thermal process.

The powder mixture according to the invention may also contain small quantities of contaminants which are introduced by the starting materials and/or by process contaminants. Said contaminants are present in a quantity of less than 1 wt. %, generally of less than 0.1 wt. %.

In a preferred embodiment of the invention, the content of the zinc/titanium mixed oxide particles in the powder mixture may amount to at least 50 wt. %. A zinc/titanium mixed oxide content of at least 80 wt. %. may be particularly preferred.

The zinc/titanium mixed oxide particles may preferably have a composition (ZnO)₁₋ₓ(TiO₂)ₓ, where 0.05<x<0.80.

The zinc/titanium mixed oxide particles may be amorphous or crystalline. Crystalline zinc/titanium mixed oxide particles may be preferred for the purposes of the invention. Crystalline means that defined reflections are observable in an X-ray diffractogram, the width of which is determined by the size of the primary particles.

It may furthermore be advantageous if the isoelectric point of the powder mixture according to the invention is between that of zinc oxide and that of titanium dioxide. The isoelectric point of zinc oxide is at approx. 9.2, that of titanium dioxide at approx. 5 to 6.

The titanium dioxide particles of the powder mixture according to the invention may have rutile, anatase and brookite modifications, the ratio of which to one another is not limited. Preferably, however, the proportion of the rutile modification of the titanium dioxide particles of the powder mixture according to the invention may amount to at least 1%, relative to the sum of rutile and anatase modification.

In a preferred embodiment, the powder mixture according to the invention may have a BET surface area which is between 1 and 100 m²/g. The range may particularly preferably be between 5 and 40 m²/g. The BET surface area is determined according to DIN 66131.

The chlorine content of the powder mixture according to the invention may, if desired, be less than 500 ppm. In particular embodiments it may be less than 100 ppm.

The invention also provides two processes for the production of the powder mixture according to the invention.

The first process is characterised in that an aerosol, which contains a zinc compound, is homogeneously mixed with a mixture containing a titanium compound, optionally an inert gas, a fuel gas and a gas containing free oxygen in a mixing chamber of a burner as is used for the production of pyrogenic oxides, the mixture of all the components is ignited at the mouth of the burner and combusted in a cooled flame tube, then the resultant solids are separated from the gaseous reaction products, optionally purified, and thermally treated.

The composition of the powder mixture may be varied by modifying the flame parameters and the thermal post-treatment.

The zinc and titanium compound may preferably be present in a ratio such that the powder mixture according to the invention contains between 20 and 95 wt. % of zinc oxide.

Titanium tetrachloride may preferably be used as the titanium compound.

The aerosol may preferably be produced by atomisation by means of a two-fluid nozzle or by an aerosol generator.

Another process for the production of the powder mixture according to the invention is characterised in that a titanium dioxide powder is dispersed in the presence of a solution of a zinc compound, wherein the ratio of titanium dioxide and zinc salt corresponds to the subsequently desired ratio of titanium dioxide and zinc oxide in the final product, the mixed oxide particles being calculated separately as titanium dioxide and zinc oxide, then the solvent is removed by evaporation and the residue is thermally treated.

In both processes according to the invention, thermal treatment may preferably proceed at temperatures of 400 to 600° C. over a period of 0.5 to 8 hours.

Selection of the zinc salt is not restricted in either of the processes according to the invention. Zinc chloride, zinc nitrate and/or organozinc compounds such as zinc acetate may, for example, be used.

The solution of the zinc compound may be an aqueous or organic solution. An aqueous solution is preferred for the purposes of the invention.

Pyrogenically produced titanium dioxide powder, for example titanium dioxide P 25 from Degussa, may preferably be used as the titanium dioxide powder.

The invention also provides a sunscreen preparation which contains the powder mixture according to the invention in a proportion of 0.01 and 25 wt. %. The sunscreen preparation according to the invention may additionally be used in mixtures with known inorganic UV-absorbing pigments and/or chemical UV filters.

Known UV-absorbing pigments which may be considered are titanium dioxides, zinc oxides, aluminium oxides, iron oxides, cerium oxides, zirconium oxides, barium sulfate or mixtures thereof.

Chemical UV filters which may be considered are any water- or oil-soluble UVA and UVB filters known to the person skilled in the art, the following being mentioned by way of non-limiting examples: sulfonic acid derivatives of benzophenones and benzimidazoles, derivatives of dibenzoylmethane, benzylidenecamphor and the derivatives thereof, derivatives of cinnamic acid and the esters thereof, or esters of salicylic acid. Selected examples may be: 2-hydroxy-4-methoxybenzophenone, 2-hydroxy-4-methoxybenzophenone-5-sulfonic acid, 2-hydroxy-4-methoxybenzophenone-5-sulfonate sodium salt, dihydroxydimethoxybenzophenone, dihydroxydimethoxybenzophenone sulfonate sodium salt, tetrahydroxybenzophenone, p-aminobenzoic acid, ethyl p-aminobenzoate, glyceryl p-aminobenzoate, amyl p-dimethylaminobenzoate, octyl p-dimethylaminobenzoate, ethyl p-methoxycinnamate, isopropyl p-methoxycinnamic acid ester, octyl p-methoxycinnamic acid ester, 2-ethylhexyl-p-methoxycinnamic acid ester, p-methoxycinnamic acid ester sodium salt, glyceryl di-p-methoxycinnamic acid ester mono-2-ethylhexanoate, octyl salicylate, phenyl salicylate, homomenthyl salicylate, dipropylene glycol salicylate, ethylene glycol salicylate, myristyl salicylate, methyl salicylate, 4-t-butyl-4-methoxydibenzoylmethane and 2-(2′-hydroxy-5′-methylphenyl)benzotriazole. Among these, 2-ethylhexyl-p-methoxycinnamic acid ester and 4-tert.-butyl-4′-methoxydibenzoylmethane are preferred by virtue of the UV protection they provide and their skin compatibility.

The sunscreen preparation according to the invention may furthermore contain solvents known to the person skilled in the art, such as water, mono- or polyhydric alcohols, cosmetic oils, emulsifiers, stabilisers, consistency regulators such as carbomers, cellulose derivatives, xanthan gum, waxes, bentones, pyrogenic silicas and further substances conventional in cosmetics such as vitamins, antioxidants, preservatives, dyes and fragrances.

The sunscreen preparation according to the invention may typically assume the form of an emulsion (O/W, W/O or multiple), aqueous or aqueous-alcoholic gel or oil gel and be offered for sale in the form of lotions, creams, milk sprays, foam, sticks or other usual forms.

The general preparation of sunscreen preparations is furthermore described in A. Domsch, “Die kosmetischen Praparate”, Verlag fur chemische Industrie (ed. H. Ziolkowsky), 4th edition, 1992 or N. J. Lowe and N. A. Shaat, Sunscreens, Development, Evaluation and Regulatory Aspects, Marcel Dekker Inc., 1990.

The invention also provides the use of the powder mixture according to the invention as an adsorbent for UV radiation.

### EXAMPLES

Remission is determined by means of a Perkin-Elmer model P554 spectrometer with remission sphere.

The BET surface area is determined according to DIN 66131.

### Example 1

0.60 kg/h of TiCl₄ are volatilised in a vaporiser at approx. 150° C. and the vapour is passed into the mixing chamber of a burner by means of 0.14 Nm³/h of nitrogen. In this chamber, the stream of gas is mixed with 1.4 Nm³/h of hydrogen and 2.0 Nm³/h of dried air and supplied to the flame through the mouth of the burner. The burner consists of two concentric tubes, in the middle of which there is additionally located a two-fluid nozzle for atomising liquids by means of a stream of gas, said nozzle finishing at the level of the mouth of the burner. 0.2 Nm³/h of hydrogen are passed as jacket gas through the outer tube of the burner. 330 ml/h of an aqueous zinc acetate solution (400 g/l) are pumped by means of a gear pump through the liquid tube of the two-fluid nozzle (internal diameter 0.2 mm), the solution being atomised by means of 550 l/h of air. The gases and atomised liquid are combusted in the reaction chamber and cooled to approx. 110° C. in a downstream coagulation section. The resultant powder is then deposited in a filter. The powder mixture according to the invention is obtained in a subsequent heat treatment step at 600° C. for a duration of 40 minutes.

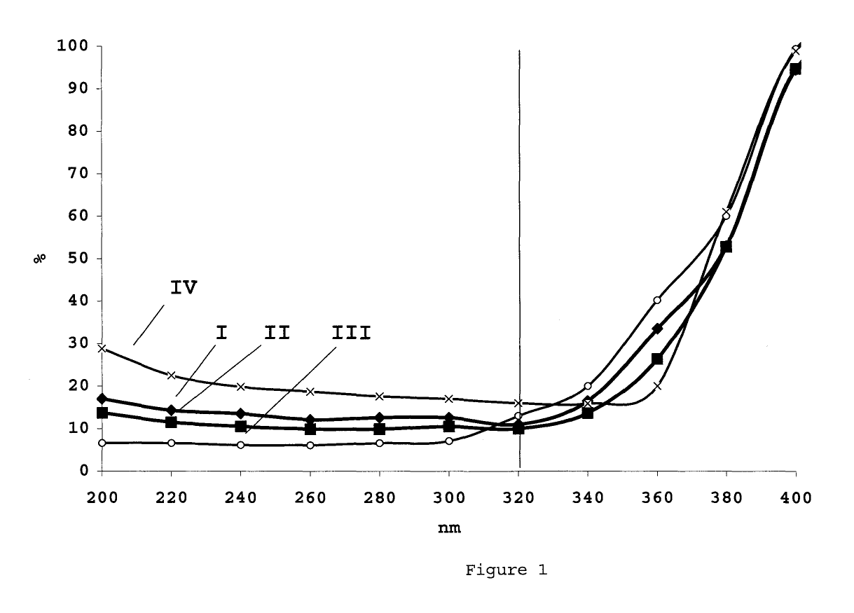

X-ray diffraction analysis of the powder mixture before heat treatment reveals that it comprises a mixture of titanium dioxide and zinc oxychloride (Zn₂OCl₂). X-ray diffraction analysis after heat treatment reveals a mixture of zinc/titanium mixed oxide, titanium dioxide, zinc oxide with a BET surface area of 40 m²/g, a pH value (4% aqueous dispersion) of 6.45, a bulk density of 290 g/l and a tamped density of 340 g/l. FIG. 1 shows the remission of this powder mixture.

The titanium dioxide has a rutile/anatase ratio of 30:70 before heat treatment and of 45:55 after heat treatment.

### Example 2A

Pyrogenically produced titanium dioxide (P25, Degussa) is dispersed by means of a laboratory stirrer in a zinc nitrate solution in 100 ml of water. The water is then removed at 90° C. and the residue treated at 550° C. over a period of 3 hours. Heat treatment at 550° C. is then performed for 3 hours.

Table 1 shows the quantities used and the physicochemical values obtained in Examples 2A-D. The stated values for TiO₂ and ZnO were determined by X-ray fluorescence analysis and contain zinc/titanium mixed oxide. X-ray diffraction analysis reveals the presence of a mixture of zinc/titanium mixed oxide, titanium dioxide and zinc oxide.

FIG. 1 shows the remission (in %) of the powder from Example 1 (denoted I) and Example 2C (II) in comparison with a pyrogenically produced titanium dioxide (P25, Degussa, III) and a zinc oxide (Nanox 100, Elementis, IV) as a function of wavelength. The powder mixtures (I) and (II) according to the invention exhibit remission which, in the UV range from 320 to 400 nm, is lower than that of titanium dioxide and, in the UV range below 320 nm, is lower than that of zinc oxide.

TABLE 1
Quantities used and physicochemical values of
Examples 2A-D

| | Starting substances | | | Product | | |
|---|---|---|---|---|---|---|
| | TiO₂ | Zinc salt | | TiO₂ | ZnO | BET |
| Example | g | | g | [wt. %] | [wt. %] | [m²/g] |
| 2-A | 10 | ZnCl₂ | 5 | 87.7 | 12.3 | 25 |
| 2-B | 5 | ZnNO₃⁽*⁾ | 10 | 68.8 | 31.2 | 19 |
| 2-C | 5 | ZnNO₃⁽*⁾ | 30 | 33.5 | 66.5 | 14 |
| 2-D | 5 | ZnNO₃⁽*⁾ | 50 | 22.6 | 77.4 | 8 |

⁽*⁾as hexahydrate

The powder mixture from Example 2-C has an isoelectric point of 8.3.

### Example 3

### Sunscreen Preparation

A sunscreen preparation was produced on the basis of the following formulation comprising 4 wt. % of the particles according to the invention according to Example 2-C (values in brackets in wt. %). Phase A: Isolan GI 34 (3.0), castor oil (1.2), Tegesoft OP (10.0), Tegesoft Liquid (5.0), glycerol 86% (3.0), phase B: Paracera W80 (1.8), isohexadecane (5.0), phase C: powder mixture according to the invention according to Example 2-C (4.0), phase D: magnesium sulfate (0.5), deionised water (66.5).

Phase A is heated to 70° C. in a mixer. After being melted on a magnetic hot plate at 80° C., phase B is added to phase A. Phase C is stirred into the oil phase at approx. 300 rpm and under a vacuum. Phase D is also heated to 70° C. and added under a vacuum to the mixture of A-C.

1. A process for production of a powder mixture including zinc/titanium mixed oxide particles, titanium dioxide particles and zinc oxide particles, comprising:
homogenously mixing an aerosol containing a zinc compound with a mixture containing a titanium compound, optionally an inert gas, a fuel gas and a gas containing free oxygen in a mixing chamber of a burner as used for the production of pyrogenic oxides;
igniting the mixture of all components at the mouth of the burner and combusting in a cooled flame tube;
separating resultant solids from gaseous reaction products;
optionally purifying the resultant solids; and
thermally treating the resultant solids;
wherein:
the zinc/titanium mixed oxide particles have a composition according to the formula (ZnO)1-x(TiO2)x, where 0.05<x<0.80; and
the powder mixture exhibits remission which, in the UV range from 320 to 400 nm, is lower than that of titanium dioxide and, in the UV range below 320 nm, is lower than that of zinc oxide.

2. The process of claim 1, wherein the zinc compound and the titanium compound are present in a ratio such that the reaction product contains between 20 and 95 wt. %. of zinc oxide.

3. The process of claim 1, wherein titanium tetrachloride is used as the titanium compound.

4. The process of claim 1, wherein the aerosol is produced by atomisation by means of a two-fluid nozzle or by an aerosol generator.

5. The process of claim 1, wherein the thermal treatment proceeds at temperatures of 400 to 600° C. over a period of 0.5 to 8 hours.

6. The process of claim 1, wherein zinc chloride, zinc nitrate and/or organozinc compounds are used as the zinc compound.
